SEQUENCE LISTING (2 sequences)

SEQ ID NO: 1 - DNA, 2152 bp, Cryptomeria japonica
  misc_feature LOCATION: (1919)..(1919): n is a, c, g, or t
agagagcaagtcatggcaatggcaatctcaggaatggcgggactttctgctgcttccatggctgctgtcaactactcatcttcagaatctcaacttcttgctcctgttgccccgcgttctggtaaggtttcatttcatggcgataatttcaccggtcctggaagtgtgtgtacacgcacttgccttgtcgtgagaaagattggctttttcatgtcttaagttactgtttattcaacttcggcaactgggtaccagtgtttctaccttgttttgtccagttccatacatggggcattcaagaactgatttcagtagtcactgttggttttctttttctttttttctcggttcggcatagttcattgctaattttgtagattttgtgacttaaatgtattccatattggaaataccaaaattgagcgtaatacttgtttgttttagataacaaaatctaatattttattgaaattgggtcaggccagttccttggaaatagactccgaacgggttgcaaaatgcaactgcctaagaagaggttcaggagcactttatgcgtgaacagtgttgcgagtgaagttttatcgactaagcaggtaagttttgcttctgtcaaaggttttcattttgttctgccaacaatgatgtagtttatccacggtacaatgcttctaaaatacgactctgtaatcttggatctccgagaatgaacttttaattcactgaaatagcatcagttaagctctttgataggatcacagtttaaaatgcccgaaaacaattgaaggtatttcttgcattggatggtttttgtaagtcaattaaaatgtttttatcaattatgtggctaccataccattttttgtcttctaagtatgttccatagcaagtagttatatttaatcgatagatcaattgctatctggatgaaaggctaagatccatagcgcacagttgtatttagtctgatagtgcaagttgtatctggttgaaaggctacgaaggctgtatccaaggacaatcagcgacctgtgtatccttttgcagcaattgttggtcaggatgagatgaaattatgccttttactgaatgtgattgacccaaaaattggaggggttatgataatgggtgaccgtggtacaggcaaatcaaccacagtgagatcacttgtggacttgcttccagaaattacagtggtttctggtgatccgttcaattctgatccttatgatccggaatctatgagcgaggaggttcggcaaagagttgaaagagaagaagaacttcctgtcacaatgacaaaaatctgtatggtagatttgccattgggtgccactgaggatagggtttgtggaactattgatattgagaaggcacttacagagggagttaaggcatttgagcctggtcttcttgcaaaggctaacaggggaattctgtatgttgatgaggttaatcttcttgatgatcatctagtagatgtactattagattctgcagcttctggatggaacaccgtggagagagaagggatttcaatctcccatccctctcgttttattctcattggttctggaaatccagaggaaggtgagcttcgtccgcaactgttggacagatttggtatgcatgcccaggttgggacagtaagagatgcggagttaagggtcaagattgtggaggagagagcaagttttgacagagaccaaaaggcttttcgagagaaatataagcaagagcaggaaaagcttcagagtcagatttatgaggctagggagcggctttctagagttcaaatggatcatggcattcgtgttaagatatcccaagtatgttctgagctgaatgtggatggcttgagaggtgatatagtgactaacagggctgccaaggctttagctgctctgaaaggaagagangaggtacgtcccgaagatgtgaagattgtcatcccaaattgcttgagacaccgtctccgaaaagatccattggaatccattgactcaggtttgcttgttgaagaaaaattctacgaggtatttggatgaacagggaagacagccctttgctggttctgttcagcatgctgactctttttatgatcagccaaggatttgtgtgtgcaaatgtgtcaacgtgttatagctttctg

SEQ ID NO: 2 - DNA, 6154 bp, Oryza sativa
agggggatggggaacaggataggggggaggaggaaggcgggggtggaggagaggtacacgaggccgcaggggctgtacgagcacagggacatcgaccagaagaagctccggaagctgatcctcgaggccaagctcgcgccgtgctacatgggcgccgacgacgccgccgccgccgccgacctcgaggagtgccccatctgcttcctggtataaaaccgcctccggggagatgtggcgggtggacgggaggaagagatctctcccctcttcctcacttcctcctcccgcttccttccacttcagctttttctctcttttttttttggtgggattttgtcgtaatgctactactagtacttgttgttgcgagagtagggcgtggattgcgtgtggccgggtggtggcgtggagcgtttcgattagggggttgattacttttgctcctgttcgattgtggagagtgccggtggtgtgtggccgttatggagatggatgaccacgcgcagtgttttggggcaagaatttcagtcgacattggcacggttttggggttgtttgtgttgttcttggataattagtgcggttcgtggagctgattttgcgaaatgtagtagtagtgtttgaattctttctggtagttagtagtggtatcatacttcgtatgtacattgtccgcaatgttctcctaaattgttttgttttcttaatcatagtttacttacttttattatgtaattctgagatacgtttttttgggataattatagtctaggaggttttgggtactcatttattattgtcaatattctataggcctatctattggctgcctggctgtgatgcattgcatgaaatttaatgtttactgccactcccggggttgggacattacagaccgtataggaaatttgactagtttcataaatgaataattggccaactgttctgcaatgttgggtgccttcaatttcaaatagtggatgctctatagagtacggaggtaccatggtgatatttgtttgttgtgtgtaatactttatttttggtgtattaatgctcttgggaaccgagattgagaatctattgtagtgaacttgattctgttagctgtgccagagtattttcgatgtctcttgtcaagcctgcatttatttggcacaaagtagatttttttaaaaaaaaaattctgcagctttaatgctactagcttactgcaattaatgttattactatataggaattttaatgcaattctatatggttgcaattttattgcaattctgcttcattagatctttagctcaaaatcactaaataaatttggttttagatgaaatcatgaaacttgtattataggagtatattatatttggtgttgaattatatgtttgcatttgtgctaatagtggtatttaacaattatcacttctggtatttctgagttcaaactgtttgacaaccactcctgtcctgaaatgcatgttttctttctttgtagtactacccaagtcttaaccgatcaaagtgttgctcaaaagggatatgcaccggtaatacatctatcctataactaacaattgtgttttacttgttaggaccatatacccttatattcaatggttggtttttttgcagagtgctttctccaaatgaaaccaactcacactgctcagcctacacagtatcctttgtgtcacccttgattatattttgtaaccttggcagttggcaatctatattcctttttatgaaaaaaatacttaaccaataaagatgtccattctgcaaaactcccagttatgctgtggagtatcgtggtgtaaagacaaaggaggaaaggagcatagaacaatttgtaagtaatttatattctgatgttttttttttgttttgccaaatgcatttcatttatctcacataatatgtgttacaggaagagcagaaagtcatagaagcacaaatgaggatgcgccagcaagcacttcaagatgaagaagataagatgaaaagaaaacagaacaggtgctcttctagcagaacaatcacaccgaccaaagaagtggagtatagagatatttgcagcacatccttttcaggtctgcacaggctgcaactaacatagaaatttagtaatgtaccatttcttctgcttgatgtggtaatttactatgtgttatgtctgtttcagtgccgtcataccgatgtgctgagcaagaaactgaatgctgttcatcggaaccttcatgctctgcccagactagcatgcgccctttccattctaggcataaccggtatgttattatctttttctctgagttttagggtgtcattgtagcctggttatgttgaactgcaaaccttactacacttctatgttaaataccctacctcaaccatgttgaagggaagcaaatgagaagtattgttaaaataggataaaggaaagtaacagtattatctatagaatcaaatcgataacaaagatcaaccgttggattaaattggatagtggggaccagacaccataaaacactggcatttcggacaagatcatttaattattccactttaagcttgtgcagtctgtcctgatccactgcactatttaagtggatgagaagctgtgattggagttactgttctagtgttaaggttgacttgtatcagatacaattgcctatccagtgacaaagtttgaatgtttaatttgtgaaagtgtgaatggagagaatttctgcccacatcattgttgtaccatgatagttacagtaatctttagtttatctatactctagagggataaaaacaccaaaacagtttgatcaaaattgttcgaggtaaatcctaagtataaaattgagaatggatttgtttttcaatatggttatttgttatcgacattcagtgtttttatttcacacaatggccaattccgaaaaaacaatctgggttctttttatcccactgtccataccaagttgatgtagcaagtaactactacacaaaatagtatattgtagccctttacatgcatccaatagattaagtggtcttcaatccaaggatgaaataagcaataacgtaggttccgataccaaattagcacaaaacaatgctaaatgcttttagaggaagtaaaccactcatttggttcttttacctataaacttgtgttcgcttgatgttctcaatattgtcaggctattttatagacttgaggagtaaacatttcttgatgacgttatcttctcctattgtgctagacataaaatacagtgtagcataacttgtgtactgtgagtttttttcaagtttttcttgatgctgcacaatagtatacaacaatcgtctattcagtcacacatggcacatcctcacatcaaacagaagttactttttatgatgttaatgtgtagtataacagcagaagctaaacggtagagggatacaggatactcgagtcaatatgaagttggggttccatggagtatttgaatcgaataaaacatcttgatgatcaatattggttttcatattgtttctataagctagtttcatatttttctagagaacctattattattttgcttcctgtcctcctcagaagattgacattctgtactggtagtgaatttgtgtttccctcataccctgtttgggagggtttcaacgtcagcttagtgcagtgctgcaaagtttaaactctatgaccagagcataagtatggcttaaggacttctgatgacaaagccatttttgaaacctgaagcctattttgatcaacttatgaacttatatggaaaaccagtttctgaaagaggccaaatatcaataaatggtgccttaatctcccttcagttttatagtttaccatatggatatttttcctgtactaagtgtttcttgcttgctggtcatgataatcctttatgctgttgagatgaaaatggtttctaaaattatgaaactgtgtgtttccccttacagtgatgataacattgacatgaatatagaggatatgatggttatggaagcgatttggcgttccattcaggttagtagttttctcactggtccttacatgagattattgataatatgcatatggcatactgtggataatattataaaagctgatttgttgggtcaggtaatggtttgtaaatgagctgcacaaaaatagattgttatttgcattgaacatgaaagtttgctaatctcctggcatgcttgttgaattcagagcattttgcgagttctgataaatgcaagatctagtctgcacaagcagaacctgtattgttaagtgttcaataataaaaaaaaatgtcaattaaattgttgagaagtgtattggcgatagtggataatgtaggatatgtttggtttgagggaccaacatcagttagttcatcattgcctcattcctcaggcacatgctagtggaaaaatgagtaatgcagtattcatgccaaaatcatcctagatggtgtgattcctcaaacaaaacaggccagtagatgatggggttggtaagataagttatctttttgtcattttacttgagcagtactgcgcttggcacaaaaatatttctggtcattttgtgagaacaagaggacttttcacattcctgcattcctgaagggaacttattatctaacagcattgctaaactcagctcttcccatgcccttagccatcaaattgatttgttatgatggttgcattgttagttttgcggtcccaattattagccacttaacgttcaggtcaggaactaatggtgttttttttctccgtcctttgtgttatactacctccatcctaaaataagtgcagccatgagtttccgcgcccaactttgatcgtccgttttatttgaaatttttttataattagcatttttgttgttataagatgataaaacatgaatagtactttactcgtgacttatgtttttgattttttcaaaaaaatttcaaataagacgaatggtcaaagttgggcgctgaaaaccatggctgcacttattttgggacggaggtagtatcatttattcatttgctgttcaagctgtagctcatcgtgtactgttccccaataagttgcttgccatttggtctatgtttccagcttctttcttttgtgagcatttttccttctgtatagcttgttactagaaagatgtaaactttatgttaaatgctgcttcatctttgaatttagattcttgggaataaacaataagcaaagttcatgtattgcgtatagaaaagtctgcatatatttctatcctaatacggtcaatcttttctctgagcaggagcagggaagtatagggaatcctgtctgtggcaactttatgcctgtaactgagccatctccgcgtgaacgccagccattcgttccagctgcttctctagaaatacctcatggtggtggattttcctgtgcggttgcggcaatggctgagcaccagccacccagtatggacttctcttacatggctggcagcagcgcattcccagttttcgacatgttccggcgaccatgcaacattgctggtggaagcatgtgtaatctggagagctcaccggagagctggagcgggatagcaccaagctgcagcagggaagtggtaagagaagaaggagagtgctcggctgaccactggtcggagggtgcagaggccggaacaagctacgcgggctcagacatcgtggcagatgccgggaccatgccgcagctgcctttcgccgagaacttcgccatggcgccaagccacttccgcccggagagcatcgaagaacagatgatgttttccatggctctttctttagcagatggtcatggaagaacacactcgcaagggttggcatggttgtaggtagagcactctaattttgacgccttgctgccctctcccttgcgctgctgttgctgcccttctctcccctgcctcctgcttctgcctcctttttgccaccagctcttggcctttttgttcaccccttttttgcatgtgttttgtcgtcatggtttgatatagatccagctatagctctccattgttattgcttatatgtatgtaaaatggaatatgaggaaatagaaaaaaggaaaatgggtcaacagttcttttggcagtagg

(19) United States
(12) Patent Application Publication
(10) Pub. No.: US 2023/0174959 A1
(43) Pub. Date: Jun. 8, 2023

(54) PLANT GENOME EDITING TECHNIQUE NOT RELYING ON GENE RECOMBINATION UTILIZING CELL MEMBRANE-PERMEABLE PEPTIDE

(71) Applicants: NATIONAL UNIVERSITY CORPORATION TOTTORI UNIVERSITY, Tottori (JP); FOREST RESEARCH AND MANAGEMENT ORGANIZATION, Ibaraki (JP)

(72) Inventors: Takashi Iwasaki, Tottori-shi, Tottori (JP); Kousei OMURA, Kikugawa-shi, Shizuoka (JP); Yoshino TANAKA, Sanda-shi, Hyogo (JP); Yoshihiko NANASATO, Hitachi-shi, Ibaraki (JP)

(73) Assignees: NATIONAL UNIVERSITY CORPORATION TOTTORI UNIVERSITY, Tottori (JP); FOREST RESEARCH AND MANAGEMENT ORGANIZATION, lbaraki (JP)

(21) Appl. No.: 17/920,933

(22) PCT Filed: Dec. 17, 2021
(86) PCT No.: PCT/JP2021/046845

§ 371 Date: Oct. 24, 2022

(30) Foreign Application Priority Data
Dec. 21, 2020 (JP) 2020-211301

(51) Int. Cl. C12N 9/22; C12N 9/96; C07K 7/00

(52) U.S. Cl. CPC C12N 9/22; C07K 7/00; C12N 9/96; C07K 2319/07; C12N 2800/80

(57) ABSTRACT

The present invention provides: a complex comprising a genome editing enzyme and a cell membrane-permeable peptide(CPP), wherein the CPP is fused to the genome editing enzyme; a complex comprising a genome editing enzyme, a target gene-specific nucleic acid, and a CPP, wherein the CPP is fused to the genome editing enzyme and/or the a target gene-specific nucleic acid; the complex comprising a polycationic moiety fused to the CPP, wherein the polycation moiety is statically bound to the target gene-specific nucleic acid; a genome editing method using the complex; and a kit for genome-editing, the kit including these complexes.

### TECHNICAL FIELD

The present invention relates to a genome editing technique using a novel complex. Specifically, the present invention relates to a technique to directly transfer a genome editing enzyme and a nucleic acid into plant cells without relying on gene recombination.

This application claims priority to Japanese Patent Application No. 2020-211301, the contents of which is incorporated by reference herein in its entirety.

### BACKGROUND ART

The “genome editing technique”, which is an innovative technology to modify biological functions, has been increasingly applied to a wide range of fields from basic researches to applied researches (e.g., medicine and crop breeding). Unfortunately, conventional genome editing techniques for plants have a crucial problem of involving time-consuming and complicated operations primarily because of the reliance on gene recombination.

Such conventional plant, genome editing techniques utilize gene recombination to transfer a genome editing cassette gene (a gene encoding a genome editing enzyme and a gene synthesizing a target gene-specific nucleic acid) into a plant cell and induce genome editing. Subsequently, genome-edited cells are obtained by selecting cells that have deletion of the gene encoding the genome editing enzyme. However, it is impossible to transfer the genome editing cassette gene to plant species that are not amenable to gene recombination, and the current genome editing can be applied only to some plant species. Furthermore, in conventional techniques, the genome editing enzyme gene, which is a foreign gene, is transferred into plant cells. Unless the foreign gene is removed, the resulting plants are considered as genetically modified organisms and are extremely difficult to utilize commercially as they are. The foreign gene can be removed only by repeated cross fertilization, and removal of the genome editing cassette gene requires extremely long time.

To solve the problems of conventional plant genome editing techniques as describedabove, genome editing techniques without relying on gene recombination have been actively developed. Specifically, a technique has been proposed that induces genome editing without mediating gene recombination by transferring a genome editing enzyme in the form of a protein directly into a plant cell (Non-Patent Literature 1). However, due to highly negatively charged cell walls of plant cells, basic proteins are trapped within cell walls, and acidic proteins are repelled by cell walls in an attempt to transfer proteins into plant cells. Although particle gun and electroporation techniques can be used to transfer proteins forcedly into plant cells, such techniques require special and expensive dedicated devices. To date, there have been no versatile techniques to simply transfer proteins into plant cells with cell walls and thus no techniques to simply transfer a genome editing enzyme into plant cells. Such circumstances contribute to a great delay in modification of plant functions, particularly crop breeding, by utilizing genome editing techniques.

### CITATION LIST

### Non-Patent Literature

Non-Patent Literature 1: Svitashev S. et al., Nat. Commun. 2016; 7: 13274.

### SUMMARY OF INVENTION

### Technical Problem

There is a need for techniques that enable genome editing simply in a wide range of plant species without relying on gene recombination.

### Solution to Problem

The present inventors have intensively investigated to solve the problems and found that a complex comprising a genome editing enzyme and a cell membrane-permeable peptide (hereinafter sometimes referred to as “CPP”) and a complex comprising a genome editing cassette (a genome editing enzyme and a target gene-specific nucleic acid) and the CPP can be used to directly transfer the genome editing cassette into plant cells without gene recombination. Thus, the present invention has been accomplished.

The present invention provides the following:
(1) A complex comprising a genome editing enzyme and a CPP, wherein the CPP is fused to the genome editing enzyme.
(2) A complex comprising a genome editing enzyme, a target gene-specific nucleic acid, and a CPP, wherein the CPP is fused to the genome editing enzyme and/or the target gene-specific nucleic acid.
(3) The complex according to (1) or (2), wherein the CPP is covalently attached to the genome editing enzyme and/or the target gene-specific nucleic acid.
(4) The complex according to (3), wherein the CPP is covalently attached to the genome editing enzyme.
(5) The complex according to (2), wherein a polycationic moiety is fused to the CPP and is electrostatically attached to the target gene-specific nucleic acid.
(6) The complex according to (5), comprising a polycationic moiety that is covalently attached to the CPP.
(7) The complex according to (6), wherein the polycationic moiety is a polycationic peptide.
(8) The complex according to (7), wherein the polycationic peptide comprises ten or more lysine residues or ten or more arginine residues.
(9) The complex according to any of (1) to (8), wherein 80% or more of the amino acid residues composing the CPP are histidine residues, and the CPP is eight to tens of amino acids in length.
(10) The complex according to (9), wherein all of the amino acid residues composing the CPP are histidine residues.
(11) The complex according to any of (1) to (10), further comprising a signal sequence.
(12) The complex according to any of (1) to (11), further comprising a subdomain.
(13) A method for genome editing, comprising transferring the complex according to any of (1) to (12) into a cell.
(14) The method according to (13), wherein the cell is a plant cell, an algal cell, a filamentous fungal cell, or a yeast cell.
(15) A kit for genome editing, comprising the complex according to any of (1) to (12) or components thereof.
(16) The kit according to (15), wherein the kit is used for genome editing of a plant, an alga, a filamentous fungus, or a yeast.

### Advantageous Effects of Invention

The present invention enables direct transfer of a genome editing enzyme and a genome editing cassette to plant cells without gene recombination and thus also enables transfer of a genome editing enzyme and a genome editing cassette to plant species that are not amenable to gene recombination, resulting in genome editing in such plant species. The genome editing enzyme and the genome editing cassette can be transferred to plant cells simply by incubating a complex of the present invention with plant cells in a medium. The present invention achieves highly efficient genome editing. In summary, a complex of the present invention can be used to perform simple and efficient genome editing in a wide range of plant species without gene recombination. Genome-edited plants obtained from the present invention have no foreign genes and are not considered as genetically modified organisms. Therefore, genome-edited plants obtained from the present invention can be commercially used immediately and have extremely high commercial value.

The phrase “transfer a genome editing enzyme directly into plant cells” refers to transfer of a genome editing enzyme in the form of active protein into plant cells. The phrase “transfer a genome editing cassette directly into plant cells” refers to transfer of a complex of a genome editing enzyme and a target gene-specific nucleic acid in the form of active protein complex into plant cells.

### BRIEF DESCRIPTION OF DRAWINGS

FIG. 1 illustrates genome editing using a complex of the present invention, in comparison with a conventional method.

FIG. 2 shows the nucleotide sequence of the Japanese cedar magnesium chelatase gene (CjCHLI gene) used as a target gene in the Example. In this figure, “gRNA1” and “gRNA2” represent a site recognized by gRNA1 and gRNA2, respectively.

FIG. 3 shows the nucleotide sequence of the region recognized by gRNA1 in which genome editing (gene deletion) was detected in the Example. The genome-edited (gene deleted) site is indicated as “-”. The “Samples” column lists conditions under which plant cells were treated. The “Reads” column lists the number of sequences that were subjected to nucleotide sequencing and the number of sequences where genome editing (gene deletion) was confirmed to occur. The “Efficiency” column lists genome editing efficiency. The underlines indicate conditions that were significantly confirmed to induce genome editing (gene deletion).

FIG. 4 shows the nucleotide sequence of the region recognized by gRNA2 in which genome editing (gene deletion) was detected in the Example. The genome-edited (gene deleted) site is indicated as “-”. The “Samples” column lists conditions under which plant cells were treated. The “Reads” column lists the number of sequences that were subjected to nucleotide sequencing and the number of sequences where genome editing (gene deletion) was confirmed to occur. The “Efficiency” column lists genome editing efficiency. The underlines indicate conditions that were significantly confirmed to induce genome editing (gene deletion).

FIG. 5 shows the nucleotide sequence of the region recognized by gRNA2 in which genome editing (gene deletion) was detected in the Example. The genome-edited (gene deleted) sites are indicated as “-”. The “Samples” column lists conditions under which plant cells were treated. The “Reads” column lists the number of sequences that were subjected to nucleotide sequencing and the number of sequences where genome editing (gene deletion) was confirmed to occur. The “Efficiency” column lists genome editing efficiency. The underlines indicate conditions that were significantly confirmed to induce genome editing (gene deletion) .

FIG. 6 shows the nucleotide sequence of the region recognized by gRNA2 in which genome editing (gene deletion) was detected in the Example. The genome-edited (gene deleted) sites are indicated as “-”. The “Samples” column lists conditions under which plant cells were treated. The “Reads” column lists the number of sequences that were subjected to nucleotide sequencing and the number of sequences where genome editing (gene deletion) was confirmed to occur. The “Efficiency” column lists genome editing efficiency. The underlines indicate conditions that were significantly confirmed to induce genome editing (gene deletion).

FIG. 7 shows the nucleotide sequence of the region recognized by gHNA2 in which genome editing (gene deletion) was detected in the Example. The genome-edited (gene deleted) sites are indicated as “-”. The “Samples” column lists conditions under which plant cells were treated. The “Reads” column lists the number of sequences that were subjected to nucleotide sequencing and the number of sequences where genome editing (gene deletion) was confirmed to occur. The “Efficiency” column lists genome editing efficiency. The underlines indicate conditions that were significantly confirmed to induce genome editing (gene deletion) .

FIG. 8A shows the nucleotide sequence of rice E3 ubiquitin-protein ligase GW2 gene (OsGW2 gene) that was used as a target gene in the Example. In this figure, “gRNA3” represents a site recognized by gRNA3.

FIG. 8B is continued from FIG. 8A and shows the nucleotide sequence of rice E3 ubiquitin-protein ligase GW2 gene (OsGW2 gene) that was used as a target gene in the Example.

FIG. 9 shows the nucleotide sequence of the region recognized by gRNA3 in which genome editing (gene deletion) was detected in the Example. The genome-edited (gene deleted) site is indicated as “-”. The “Samples” column lists conditions under which plant cells were treated. The “Reads” column lists the number of sequences that were subjected to nucleotide sequencing and the number of sequences where genome editing (gene deletion) was confirmed to occur. The “Efficiency” column lists genome editing efficiency. The underline indicates conditions that were significantly confirmed to induce genome editing (gene deletion).

FIG. 10 shows the nucleotide sequence of the region recognized by gRNA3 in which genome editing (gene deletion) was detected in the Example. The genome-edited (gene deleted) site is indicated as “-”. The “Samples” column lists conditions under which plant cells were treated. The “Reads” column lists the number of sequences that were subjected to nucleotide sequencing and the number of sequences where genome editing (gene deletion) was confirmed to occur. The “Efficiency” column lists genome editing efficiency. The underline indicates conditions that were significantly confirmed to induce genome editing (gene deletion) .

FIG. 11 shows the nucleotide sequence of the region recognized by gRNA3 in which genome editing (gene deletion) was detected in the Example. The genome-edited (gene deleted) site is indicated as “-”. The “Samples” column lists conditions under which plant cells were treated. The “Reads” column lists the number of sequences that were subjected to nucleotide sequencing and the number of sequences where genome editing (gene deletion) was confirmed to occur. The “Efficiency” column lists genome editing efficiency. The underline indicates conditions that were significantly confirmed to induce genome editing (gene deletion).

FIG. 12 shows the nucleotide sequence of the region recognized by gRNA3 in which genome editing (gene deletion) was detected in the Example. The genome-edited (gene deleted) site is indicated as “-”. The “Samples” column lists conditions under which plant cells were treated. The “Reads” column lists the number of sequences that were subjected to nucleotide sequencing and the number of sequences where genome editing (gene deletion) was confirmed to occur. The “Efficiency” column lists genome editing efficiency. The underline indicates conditions that were significantly confirmed to induce genome editing (gene deletion) .

FIG. 13 shows the nucleotide sequence of the region recognized by gRNA3 in which genome editing (gene deletion) was detected in the Example. The genome-edited (gene deleted) sites are indicated as “-”. The “Samples” column lists conditions under which plant cells were treated. The “Reads” column lists the number of sequences that were subjected to nucleotide sequencing and the number of sequences where genome editing (gene deletion) was confirmed to occur. The “Efficiency” column lists genome editing efficiency. The underline indicates conditions that were significantly confirmed to induce genome editing (gene deletion).

### DESCRIPTION OF EMBODIMENTS

In one aspect, the present invention provides a complex comprising a genome editing enzyme and a CPP, in which the CPP is fused to the genome editing enzyme. The fusion of the CPP will be described below. Typical examples of the genome editing enzyme in the complex of this aspect include TALEN and ZFN.

In a further aspect, the present invention provides a complex comprising a genome editing enzyme, a target gene-specific nucleic acid, and a CPP, in which the CPP is fused to the genome editing enzyme and/or the target gene-specific nucleic acid.

The CPP may be fused in any manner unless the fusion prevents transfer of the complex into plant cells and genome editing. The fusion may be made, for example, by a covalent bond such as a peptide bond or by a noncovalent bond such as an electrostatic bond and a van der Waals force. When the CPP is covalently attached to the genome editing enzyme, the covalent bond may be any type of covalent bonds, typically a peptide bond.

The CPP may be located in any position relative to the genome editing enzyme. The CPP may be fused to the N-terminus of the genome editing enzyme, to the C-terminus of the genome editing enzyme, to both the N-terminus and C-terminus of the genome editing enzyme, or to any amino acid residue that is not at the N-terminus or C-terminus of the genome editing enzyme. Preferably, the CPP is fused to the N-terminus or C-terminus of the genome editing enzyme. The CPPmay be fused to the genome editing enzyme via a linker. There are various known linkers, and they may be used for the fusion. Preferable linkers do not prevent transfer of the complex of the present invention into plant cells and genome editing. When the fusion is made by a peptide bond, examples of the linker include, but are not limited to, a peptide consisting of one or several glycine residues. One genome editing enzyme may be fused to one CPP or two or more CPPs.

The CPP may be fused to a target gene-specific nucleic acid. The CPP may be fused to the 3′ or 5′ end of the nucleic acid or to any portion that is not at the 3′ end or 5′ end, for example, a sugar portion and/or a base portion of the nucleic acid. Preferably, the CPP is fused to the 3′ end of the nucleic acid. The fusion may be made, for example, by any known method such as organic synthesis. One nucleic acid may be fused to one CPP or two or more CPPs.

There are various known CPPs. CPPs to be used in the present invention may be any peptides as long as they allow direct transfer of the complex of the present they prevent genome invention into plant cells and unless editing. Examples of the CPP that can be used in the present invention include, but are not limited to, a peptide rich in basic amino acids (e.g., arginine, lysine, and histidine) or a polyhistidine.

Furtherexamples of the CPPthat can be used in the present invention include a peptide that comprises several amino acids or more, in which a half or more of the constituent amino acids are histidine residues. Such a peptide has superior cell membrane permeability.

The length of the CPP that can be used in the present invention is not limited to a particular length, and the CPP typically has several amino acids or more, for example, several amino acids of totensofamino acids. For example, the CPP that can be used in the present invention may have 6 amino acids to 40 amino acids, 7 amino acids to 30 amino acids, or 8 amino acids to 20 amino acids, for example, 6 amino acids, 7 amino acids, 8 amino acids, 9 amino acids, 10 amino acids, 11 amino acids, 12 amino acids, 13 amino acids, 14 amino acids, 15 amino acids, 16 amino acids, 17 amino acids, 18 amino acids, 19 amino acids, 20 amino acids, 21 amino acids, 22 amino acids, 23 amino acids, 24 amino acids, 25 amino acids, 26 amino acids, 27 amino acids, 28 amino acids, 29 amino acids, 30 amino acids, or more than 30 amino acids. As used herein, several amino acids mean 2, 3, 4, 5, 6, 7, 8, or 9 amino acids. As used herein, tens of amino acids mean any number of amino acids ranging from 10 to 100 amino acids.

Preferable examples of the CPP that can be used in the present invention include a peptide (polyhistidine) in which about 80%or more, more preferably about 90% or more, and even more preferably all of the constituent amino acid residues are histidine residues. The length of polyhistidine is the same as that of the CPP as described above.

Amino acid residues except histidine composing the CPP that can be used in the present invention may be any amino acid residues. Preferably, amino acid residues except histidine composing the CPP that can be used in the present invention are basic amino acid residues such as arginine and lysine or an amino acid residue that has properties similar to those of histidine. Amino acid residues composing the CPP that can be used in the present invention may be a natural amino acid residue, an unnatural amino acid residue, a modified amino acid residue, or a synthetic amino acid residue. Those skilled in the art may appropriately synthesize or modify amino acids.

The CPP that can be used in the present invention can be prepared by peptide synthesis such as Fmoc solid-phase synthesis or by a known method such as gene recombination.

A highly cell membrane-permeable peptide (a peptide as described above) can be used to increase transfer efficiency of a complex of the present invention into plant cells, resulting in increased genome editing efficiency.

The genome editing enzyme to be used in the present invention may be any genome editing enzyme and is not limited to a particular enzyme. There are various known genome editing enzymes. Examples of the genome editing enzyme that can be used in the present invention include, but are not limited to, Cas family nucleases such as Cas9, Casl2, Cas13, Casϕ, and TiD; nucleases such as TALEN and ZFN; and deaminases such as Activated-induced cytidine deaminase (AID) and Target-G. As used herein, include a wild-type genome the genome editing enzyme editing enzyme and a mutant genome editing enzyme. The mutant genome editing enzyme includes both a natural mutant and an artificial mutant. Editing efficiency of the mutant genome editing enzyme may be increased, decreased, or lost, compared to the original enzyme. A method of producing the mutant genome editing enzyme is known and includes, but is not limited to, gene recombination, peptide chemical synthesis, and chemical modification. The genome editing enzyme may cleavage a single strand DNA or a double strand DNA.

In a particular specific example of the present invention, the genome editing enzyme is Cas9. In a more particular specific example of the present invention, the genome editing enzyme is Cas9-bound AID that has a cleaving activity modulated from modified functions.

When the genome editing enzyme is TALEN or ZFN, these proteins are designed to bind to a target gene. Such designs can be performed by a method known to those skilled in the art.

The target gene-specific nucleic acid can direct a genome editing enzyme to a site where a mutation is desired in a target gene. Typical examples of the target gene-specific nucleic acid include but are not limited to a guide RNA (gRNA). The target gene-specific nucleic acid can be designed and prepared by a known method based on the nucleotide sequence of the target gene. gRNA can be designed with known software programs such as CRISPERdirect, CRISPR-P2.0, Geneious, and ApE. Preferably, the target gene-specific nucleic acid is specific to a genome editing enzyme and forms a complex with the genome editing enzyme by incubating the nucleic acid with the genome editing enzyme. Examples of the nucleic acid and the genome editing enzyme include, but are not limited to, gRNA and Cas9.

Plants that undergo genome editing in the present invention include all kinds of plants. Plants include seed plants, pteridophytes, and bryophytes. Seed plants include angiosperms and gymnosperms. Angiosperms include dicotyledons and monocotyledons. Dicotyledons include sympetalous plants and sympetalous plants. Examples of sympetalous plants include, but are not limited to, plants belonging to the family Asteraceae, Ericaceae, Lamiaceae, Solanaceae, Convolvulaceae, Pedaliaceae, Primulaceae, and Campanulaceae. Examples of sympetalous plants include, but are not limited to, plants belonging to the family Brassicaceae, Rosaceae, Theaceae, Caryophyllaceae, Portulacaceae, Myricaceae, Cucurbitaceae, Rutaceae, Apiaceae, Leguminosae, and Lauraceae. Examples of monocotyledons include, but are not limited to, the family Iridaceae, Poaceae, Juncaceae, Araceae, Zingiberaceae, Commelinaceaea, Bromeliaceae, Musaceae, Liliaceae, and Orchidaceae. Examples of gymnosperms include, but are not limited to, plants belonging to the family Cupressaceae, Pinaceae, Taxodiaceae, Taxaceae, Ginkgoaceae, and Cycadaceae. Examples of pteridophytes include, but are not limited to, osmund, bracken fern, ball fern, male fern, field horsetail, and equisetum. Examples of bryophytes include, but are not limited to, liverwort, Polytrichum juniperinum, Schistostega pennata, and sphagnum moss.

The present invention may be used to transfer a genome editing enzyme to various plants such as edible plants, garden plants, ornamental plant, trees for building, roadside trees, and shelterbelt trees to perform genome editing. Application examples of the genome editing include, but are not limited to, cultivar improvement and genetic study.

The complex of the present invention may be used to perform genome editing in animals, microorganisms such as filamentous fungi, yeast, bacteria, and actinomycete, and algae, as well as in plants.

The complex can be produced by any known method such as chemical synthesis or gene recombination. For example, the complex can be obtained by producing a fusion of a genome editing enzyme and a CPP via gene recombination with a fusion of a DNA encoding the genome editing enzyme and a DNA encoding the CPP; and incubating the produced fusion with a target gene-specific nucleic acid. The incubation is typically performed in an aqueous solution at room temperature or about 37° C. The aqueous solution may be a buffer. If necessary, the complex may be purified by a known means such as column chromatography.

Examples of the complex of the present invention that has a CPP fused by a noncovalent bond include a complex in which a polycationic moiety that is fused to the CPP and is electrostatically attached to a target gene-specific nucleic acid. In the complex, the genome editing cassette is fused to the CPP through the polycationic moiety.

The polycationic moiety is a moiety that has two or more positively charged groups under physiological conditions and can be electrostatically attached to a target gene-specific nucleic acid. The physiological conditions may be, for example, a pH at which plant cells can survive or grow or a pH found in plant cells.

The phrase electrostatically attached refers to attachment of a negatively charged nucleic acid to a positively charged polycationic moiety under physiological conditions by electrostatic attractive forces.

The polycationic moiety and the CPP may be fused together in any manner unless the fusion prevents transfer of the complex of the present invention into plant cells and genome editing. The fusion may be made, for example, by a covalent bond, an electrostatic bond, or a van der Waals force. Typically, the polycationic moiety and the CPP is fused together by a covalent bond. Typical examples of the covalent bond include a peptide bond. The polycationic moiety may be located in any position relative to the CPP. The polycationic moiety may be attached to the N-terminus of the CPP, to the C-terminus of the CPP, to both the N-terminus and C-terminus of the CPP, or to any amino acid residue that is not at the N-terminus or C-terminus of the CPP. The polycationic moiety may be attached to the CPP via a linker. There are various known linkers, and they may be used for the fusion. Preferable linkers do not prevent transfer of the complex of the present invention into plant cells and genome editing. When the fusion is made by a peptide bond, examples of the linker include, but are not limited to, a peptide consisting of one or several glycine residues. One CPP may be fused to one polycationic moiety or two or more polycationic moieties. Also, one polycationic moiety may be fused to one CPP or two or more CPPs.

The polycationic moiety may be any types unless it prevents transfer of the complex of the present invention into plant cells and genome editing. Examples of the polycationic moiety include, but are not limited to, a peptide positively charged under physiological conditions (preferably a polycationic peptide), an oligosaccharide, and a cationic polymer. The peptide and oligosaccharide may be a wild-type, mutant, or modified peptide and oligosaccharide. The mutant peptide, mutant oligosaccharide, and those modified therefrom have an ability to electrostatically attach to a target gene-specific nucleic acid, in which the ability is equal to or more than that of the original peptide and oligosaccharide. The cationic polymer may be naturally occurring or chemically synthesized.

The polycationic peptide is a peptide that has two or more amino acid residues positively charged under physiological conditions. Such a peptide is known. Examples of the polycationic peptide include, but are not limited to, a peptide rich in basic amino acids (e.g., lysine, arginine, and histidine). The length of the polycationic peptide is not limited to a particular length unless it prevents transfer of the complex into plant cells and genome editing. The polycationic peptide is typically several amino acids to tens of amino acids in length. For example, the polycationic peptide may have 6 amino acids to 40 amino acids, 8 amino acids, 9 amino acids, 10 amino acids, 11 amino acids, 12 amino acids, 13 amino acids, 14 amino acids, 15 amino acids, 16 amino acids, 17 amino acids, 18 amino acids, 19 amino acids, 20 amino acids, or more than 20 amino acids. Examples of the polycationic peptide include a peptide consisting of a lysine residue and/or an arginine residue. Specific examples of the peptide consisting of a lysine residue and/or an arginine residue include K8, K9, K10, K11, K12, R8, R9, R10, R11, and R12. Further specific examples of the polycationic peptide include a peptide consisting of several KH repeat sequences. The polycationic peptide is not limited to the examples as described above. Amino acid residues composing the polycationic peptide that can be used in the present invention may be a natural amino acid residue, an unnatural amino acid residue, a modified amino acid residue, or a synthetic amino acid residue. Those skilled in the art may appropriately synthesize or modify amino acids.

Examples of the positively charged oligosaccharide include, but are not limited to, a polymer of a hexosamine such as glucosamine, fructosamine, galactosamine, or mannosamine, for example, chitosan. The number of sugar residues in the positively charged oligosaccharide is not limited to a particular number unless it prevents transfer of the complex of the present invention into plant cells and genome editing.

Examples of the cationic polymer include, but are not limited to, polyethylenimine, polypropyleneimine, poly(β-amino ester), polylactic/polyglycol ic acid, and 2-hydroxypropyl methacrylamide. The length of the cationic polymer is not limited to a particular length unless it prevents transfer of the complex of the present invention into plant cells and genome editing.

The polycationic peptide, positively charged oligosaccharide, and cationic polymer can be produced by a method known to those skilled in the art or extracted from natural products.

The complex can be produced by a known method. For example, the complex can be produced by incubating (i) a complex (genome editing cassette) obtained from incubation of a genome editing enzyme with a target gene-specific nucleic acid with (ii) a fusion of a polycationic moiety and a CPP; and utilizing negative charge of the nucleic acid and positive charge of the polycationic moiety to cause electrostatic binding. The polycationic moiety to cause electrostatic binding. The incubation is typically performed in an aqueous solution at room temperature or about 37° C. The aqueous solution may be a buffer. The fusion of the polycationic moiety and CPP can be produced by a known method, for example, peptide synthesis such as Fmoc method or gene recombination. If necessary, the complex may be purified by a known means such as column chromatography.

Specific examples of the fusion of the polycationic peptide and CPP include, but are not limited to, K10(G)H8, K10(G}H12, K10(G)H16, K10(G)H20, and R10(G)H20, wherein (G) indicates that a glycine residue may be present or absent.

The complex of the present invention may further comprise a signal sequence. The signal sequence is also referred to as signal peptide. When the complex of the present invention has a signal sequence, the complex of the present invention can be localized to a desired intracellular compartment. There are various known types of signal sequences. Examples of the signal sequence include, but are not limited to, a nuclear localization signal sequence (NLS), a mitochondrial localization signal sequence (MLS), and a chloroplast localization signal sequence (CLS). The signal sequence can be selected depending on an intracellular compartment to which it is desired to localize a complex of the present invention and can be fused to the complex. The complex of the present invention further comprising a signal sequence can be used to perform genome editing in a desired intracellular compartment. For example, a a complex of the present invention comprising a nuclear localization signal can be used to perform genome editing in a nucleus without gene recombination. A complex of the present invention comprising a mitochondrial localization signal sequence can be used to perform genome editing in a mitochondrion without gene recombination. A complex of the present invention comprising a chloroplast localization signal sequence can be used to perform genome editing in a mitochondrion without gene recombination.

The signal sequence may be fused to any part of a complex of the present invention such as a genome editing enzyme, a target gene-specific nucleic acid, or a subdomain (described below). The signal sequence may be fused in any manner as long as a complex is localized to a desired intracellular compartment and unless the fusion prevents transfer of the complex into plant cells and genome editing. The fusion may be made, for example, by a covalent bond such as a peptide bond or by a noncovalent bond such as an electrostatic bond and a van der Waals force. Typically, the signal sequence is fused to a complex by a covalent bond. When the signal sequence is covalently attached to the genome editing enzyme, the covalent bond may be any type of covalent bonds, typically a peptide bond.

The signal sequence may be located in any position relative to the genome editing enzyme. The signal sequence may be fused to the N-terminus of the genome editing enzyme, to the C-terminus of the genome editing enzyme,to both the N-terminus and C-terminus of the genome editing enzyme, or to any amino acid residue that is not at the N-terminus C-terminus of the genome editing enzyme. Alternatively, the signal sequence may be inserted into the amino acid sequence of the genome editing enzyme, Preferably, the signal sequence is fused to the N-terminus or C-terminus of the genome editing enzyme. More preferably, the signal sequence is fused to the N-terminus of the genome editing enzyme. The signal sequence may be fused to the genome editing enzyme via a linker. There are various known linkers, and they may be used for the fusion. Preferable linkers allow localization of the complex to a desired intracellular compartment and do not prevent transfer of the complex into plant cells and genome editing. When the fusion is made by a peptide bond, examples of the linker include, but are not limited to, a peptide consisting of one or several glycine residues. One genome editing enzyme may be fused to one signal sequence or two or more signal sequences.

The signal sequence may be fused to a subdomain. The signal sequence may be fused to the N-terminus or C-terminus of the subdomain or to an amino acid residue that is not at the N-terminus C-terminus. The fusion may be made, for example, by any known method such as gene recombination and organic synthesis. The signal sequence may be fused to the subdomain via a linker. One subdomain may be fused to one signal sequence or two or more signal sequences.

The signal sequence may be fused to a target gene-specific nucleic acid. The sequence may be fused to the 3′ or and 5′ ends, for example, a sugar portion and/or a base portion of the nucleic acid. Preferably, the signal sequence is fused to the 3′ end of the nucleic acid. The fusion may be made, for example, by any known method such as organic synthesis. The signal sequence may be fused to the nucleic acid via a linker. One nucleic acid may be fused to one signal sequence or two or more signal sequences.

The complex of the present invention may further comprise a subdomain. As used herein, the subdomain refers to a functional protein. Such a complex of the present invention can be used to perform various desired types of genome editing. The subdomain is not limited to a particular type and includes, for example, a base substitution enzyme, a DNA methyltransferase, a DNA demethylase, a transcription-activating enzyme, and an enzyme for transcriptional repression. Those skilled in the art may select appropriately a subdomain and use it for a complex of the present invention. For example, a complex of the present invention comprising a base substitution enzyme as a subdomain can be used to perform base substitution in the genome without gene recombination. A complex of the present invention comprising a DNA methyltransferase as a subdomain can be used to methylate the genome without gene recombination. A complex of the present invention comprising a DNA demethylase as a subdomain can be used to demethylate the genome without gene recombination. A complex of the present invention comprising a transcription-activating enzyme enzyme as a subdomain can be used to activate transcription of the genome without gene recombination. A complex of the present invention comprising an enzyme for transcriptional repression as a subdomain can be used to repress transcription of the genome without gene recombination.

The complex of the present invention may comprise both a signal sequence and a subdomain. Such a complex of the present invention can be used to perform various desired types of genome editing in a desired intracellular compartment. For example, a complex of the present invention comprising a nuclear localization signal sequence and a base substitution enzyme can be used to perform base substitution in the nuclear genome without gene recombination. A complex of the present invention comprising a nuclear localization signal sequence and a DNA methyltransferase can be used to methylate the nuclear genome without gene recombination. A complex of the present invention comprising a nuclear localization signal sequence and a DNA methyltransferase can be used to demethylate the nuclear genome without gene recombination. A complex of the present invention comprising a nuclear localization signal sequence and a transcription-activating enzyme can be used to activate transcription of the nuclear genome without gene recombination. A complex of the present invention comprising a mitochondrial localization signal sequence and a base substitution enzyme can be used to perform base substitution in the mitochondrial genome without gene recombination. A complex of the present invention comprising a chloroplast localization signal sequence and an enzyme for transcriptional repression can be used to repress transcription of the chloroplast genome without gene recombination.

The subdomain may be fused to any part of the complex of the present invention. Typically, the subdomain is fused to a genome editing enzyme. The subdomain may be fused to the N-terminus of the genome editing enzyme, to the C-terminus of the genome editing enzyme, to both the N-terminus and C-terminus of the genome editing enzyme, or to any amino acid residue that is not at the N-terminus or C-terminus of the genome editing enzyme. Preferably, the subdomain is fused to the N-terminus or C-terminus of the genome editing enzyme.

The subdomain may be fused to the genome editing enzyme in any manner unless the fusion prevents functions of the subdomain, transfer of the complex into plant cells, and genome editing. The fusion may be made, for example, by a covalent bond such as a peptide bond or by a noncovalent bond such as an electrostatic bond and a van der Waals force. Typically, the signal sequence is fused to the complex by a covalent bond. When the signal sequence is covalently attached to the genome editing enzyme, the covalent bond may be any type of covalent bonds, typically a peptide bond.

The subdomain may be fused to the genome editing enzyme via a linker. There are various known linkers, and they may be used for the fusion. Preferable linkers do not prevent functions of the subdomain, transfer of the complex into plant cells, and genome editing. When the fusion is made by a peptide bond, examples of the linker include, but are not limited to, a peptide consisting of one or several glycine residues. One genome editing enzyme may be fused to one subdomain or two or more subdomains.

The subdomain may be fused to a CPP. How and where the subdomain is fused to the CPP are as described for the fusion of the genome editing enzyme to the CPP.

It can be understood that when a complex of the present invention comprising a subdomain comprises a CPP fused to the subdomain fused to a genome editing enzyme, the CPP is fused to the genome editing enzyme via the subdomain. Thus, the phrase “the CPP is fused to the genome editing enzyme”, as used herein, shall include the fusion as described above.

Although transfer of the complex of the present invention into plant cells is described above, the complex of the present invention can be transferred into cells such as animal cells, filamentous fungal cells, bacterial cells, actinomycete cells, yeast cells, and algal cells as well as plant cells because of the high permeability to cells of all biological species and is useful for genome editing in a wide range of biological species. The complex of the present invention is highly permeable, in particular, to plant cells, algal cells, filamentous fungal cells, and yeast cells, which have cell walls, and thus is suitable for genome editing in these biological species.

In a further aspect, the present invention provides a method for genome editing, comprising transferring a complex of the present invention into cells. The complex of the present invention may be transferred into cells by incubating the complex of the present invention with the cells in a medium. Those skilled in the art may appropriately select or modify a method for transferring a complex of the present invention into cells, a type of medium, and incubation conditions, depending on cell types. In the genome editing method in this aspect, the cells are typically plant cells.

For genome editing of plants, a complex of the present invention can be transferred into all forms of plant cells and into all plant tissues. For example, the complex of the present invention can be transferred into leaves, stems, shoot apices, winter buds, roots, seeds, spores, pollens, cultured cells of plants, and the like. As used herein, leaves, stems, shoot apices, winter buds, roots, seeds, spores, pollens, cultured cells of plants, and the like are collectively referred to as plant cells.

In the genome editing method of the present invention, one or more target gene-specific nucleic acids may be transferred. One or more genome editing enzymes may also be transferred. In other words, one or more than one complex of the present invention may be used for genome editing.

In a further aspect, the present invention provides a kit for genome editing, comprising a complex of the present invention or components thereof. Examples of the components of the complex of the present invention include a CPP, a polycationic moiety, a CPP fused to a genome editing enzyme, and a CPP fused to a polycationic moiety. The kit may comprise components of a complex of the present invention that are combined together to form the complex of the present invention. Typically, the kit also comprises instructions. Biological species amenable to genome editing using the kit of the present invention are not limited to a particular species, as described above. The kit of the present invention is suitably used for genome editing of a plant, an alga, a filamentous fungus, and a yeast, which have cell walls.

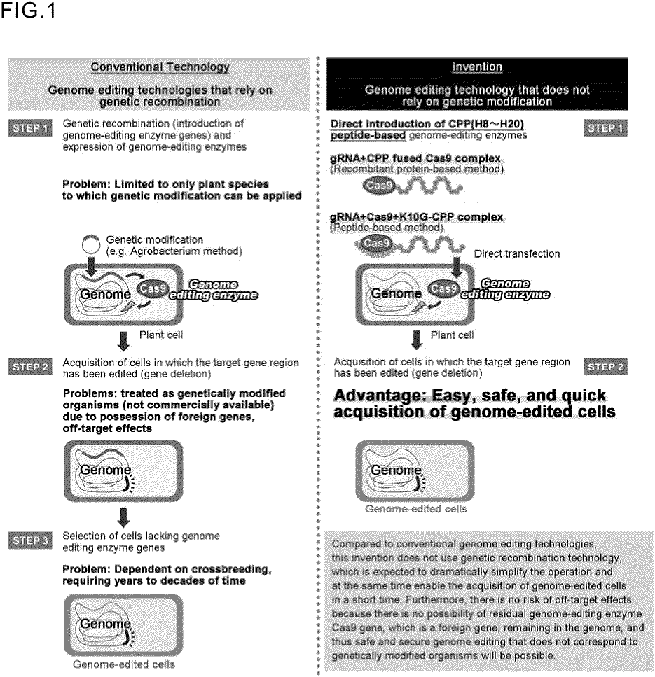

A method for genome editing by using a complex of the present invention (recombinant protein-based method or peptide-based method) will be described below and compared to a conventional genome editing method in reference to FIG. 1. The following description will specifically and clearly describe the present invention and is not limited to the scope of the present invention.

Conventional genome editing techniques have disadvantages as follows:
Convent ional genome editing techniques rely on gene recombination and can be used only for plant species amenable to gene recombination.
Plants thus genome-edited have a foreign gene and thus are considered as genetically modified organisms which are extremely difficult to use commercially.
The foreign gene can be removed only by repeated cross fertilization, and removal of the genome editing cassette requires extremely long time.

The present invention relates to a complex obtained by fusing a genome editing cassette (e.g., a genome editing enzyme Cas9 and a gRNA) to a CPP (e.g., H8 to H20 peptides) and to a method for genome editing in plants by transferring the complex directly into plant cells. The CPP can be fused by a covalent bond or a noncovalent bond (e.g., an electrostatic bond). Specific examples of the fusion technique include a method comprising preparing, via gene recombination, a recombinant protein that comprises a genome editing enzyme Cas9 fused to a CPP (recombinant protein-based method) and a method comprising electrostatically attaching a genome editing cassette to a CPP (peptide-based method). These methods can be used to prepare the complex of the present invention.

Recombinant protein-based method allows genome editing by, for example, preparing a Cas9-CPP recombinant fusion protein (e.g., a fusion protein of Cas9 and H8 to H20 CPP) in an Escherichia coli expression system, incubating the fusion protein with a gRNA for cleaving a target region to form a complex, and transferring the resulting complex into plant cells.

In peptide-based method, for example, a genome editing enzyme Cas9 is incubated with a gRNA to form a complex, and a CPP fused to a polycationic moiety (e.g., a CPP fused to K10 and H8 to H20) is electrostatically bound to the nucleic acid of the complex (by utilizing negative charge of bases in the gRNA and positive charge of lysine residues in the polycationic moiety). The resulting complex can be transferred into plant cells to perform genome editing.

Both of the methods can transfer a genome editing enzyme cassette directly into plant cells without relying on gene recombination. These methods overcome all the disadvantages of conventional genome editing techniques and can simply and quickly prepare genome-edited cells.

Unless specified otherwise, the terms as used herein are construed as commonly understood in fields such as biology, biochemistry, chemistry, pharmacy, and medicine.

A numerical value as used herein can include numerical values within a range of ±5%, ±10% or ±20% of the numerical value.

Amino acids are represented herein by known one-letter or three-letter code. When a peptide is represented herein, a number is located to the right of its amino acid represented by one-letter code. For example, H20 means a peptide consisting of 20 histidine residues. K10 means a peptide consisting of 10 lysine residues. K10H20 means a peptide having the N-terminus of a peptide consisting of 20 histidine residues bound to the C-terminus of a peptide consisting of 10 lysine residues. K10GH20 means a peptide having, in the order of the N-terminus to the C-terminus, a peptide consisting of 10 lysine residues, a peptide consisting of 1 glycine residue, and a peptide consisting of 20 histidne residues. In this specification, a peptide may comprise any bond except a peptide bond. Unless specified otherwise, bonds between amino acid residues in a peptide are peptide bonds.

As used herein, a fusion of a genome editing enzyme and a CPP is indicated by a hyphen (-). For example, Cas9-H2O means a CPP(H20) fused to the C-terminus of a genome editing enzyme Cas9. Unless specified otherwise, the bond between the genome editing enzyme and the CPP is a peptide bond.

The present invention will be further specifically described in detail in the Examples below, but the Examples do not limit the scope of the present invention.

### EXAMPLES

### 1) Test Cells

Escherichia coli strain BL21 (DE3) was used to express Cas9 and CPP-fused Cas9. Plant cells used were calli from a woody plant, Japanese cedar. (Cryptomeria japonica) and cells derived from the calli and cultured cells of a grass plant, rice (Oryza sativa). Calli from Japanese cedar were passaged onto a ½ MD agar plate every week. Cells derived from calli from Japanese cedar were suspended and tested in ½ MD liquid medium and were cultured at 25° C. while shaking at 120 rpm under protection from light. Rice cultured cells were passaged in MS liquid medium every week. Rice cultured cells were suspended and tested in MS liquid medium and were cultured at 27° C. while shaking at 120 rpm under protection from light.

### 2) Expression of CPP-Fused Cas9

Cas9-H8, Cas9-H12, Cas9-H16, and Cas9-H20, which are Cas9 proteins fused to H8, H12, H16, and H20 cell membrane-permeable peptides (CPPs) respectively, were prepared as a recombinant protein. pET24b was used as an expression plasmid, and Escherichia coli strain BL21(DE3) was used as a host strain. The recombinant protein was expressed in the host cells at 20° C. for 18 hours. A homogenate of the host cells was purified with Co (cobalt) ion-immobilized resin (GE Healthcare).

### 3) Expression of Cas9

Cas9 fused to FLAG tag (DYKDDDDK), which is a peptide tag having no cell membrane permeability, in place of CPP was prepared as a recombinant protein. pET24b was used as an expression plasmid, and Escherichia coli strain BL21 (DE3) was used as a host strain. The recombinant protein was expressed in the host cells at 20° C. for 18 hours. A homogenate of the host cells was purified with an anti-FLAG antibody-immobilized resin (MBL).

### 4) Preparation of gRNA + CPP-fused Cas9 complex (Preparation of a Complex to be Used for Recombinant Protein-Based Method)

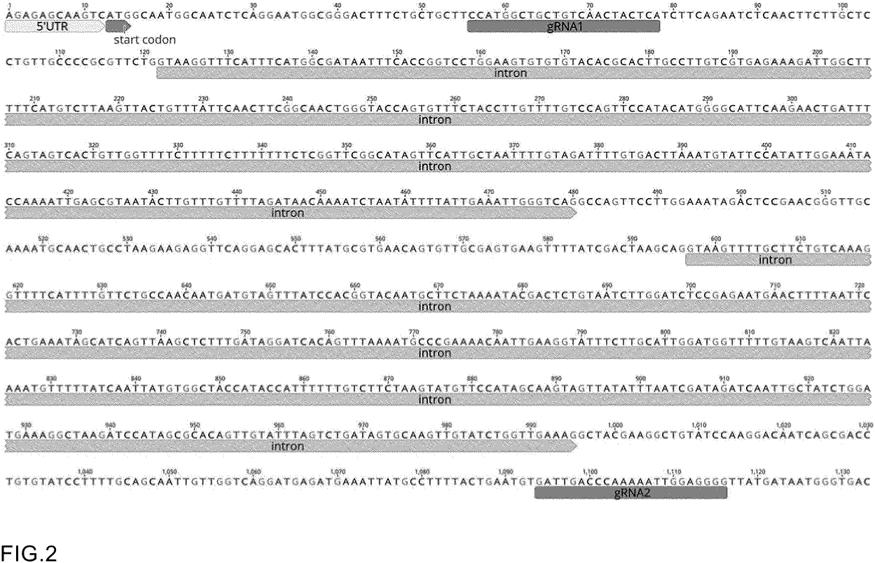

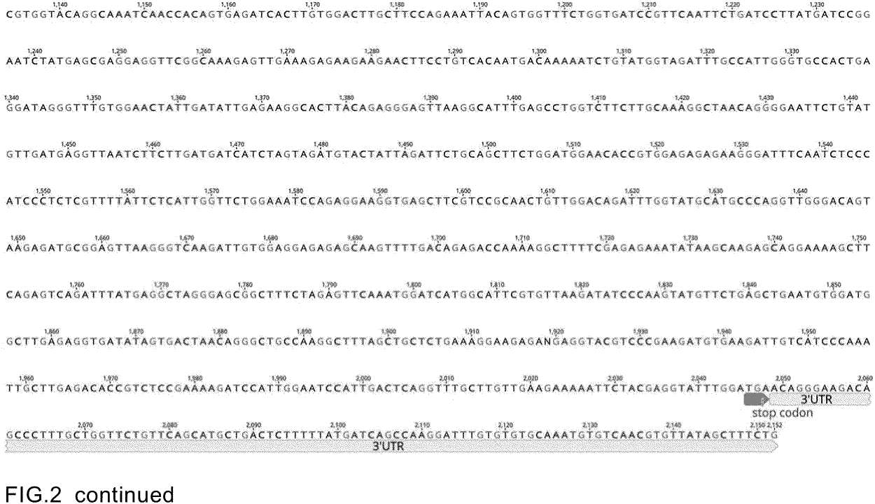

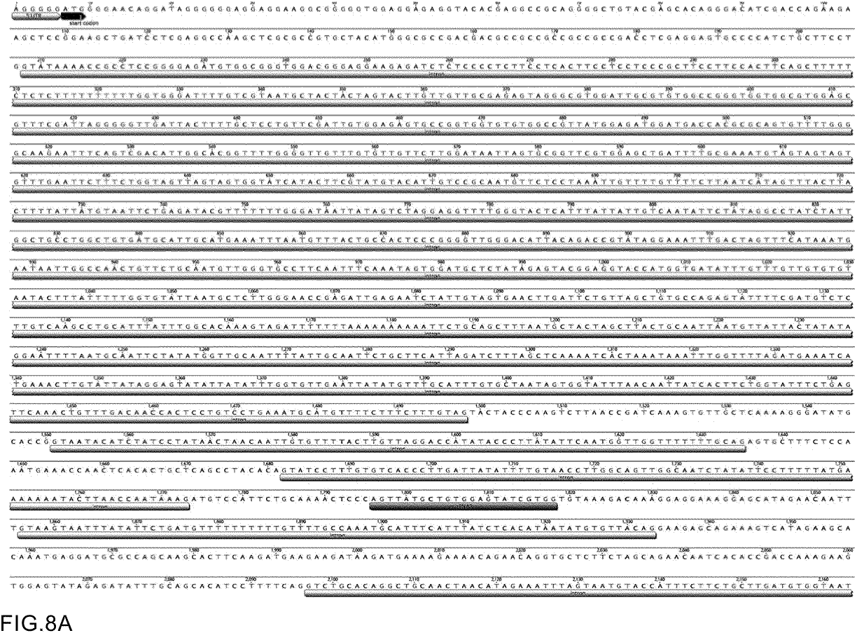

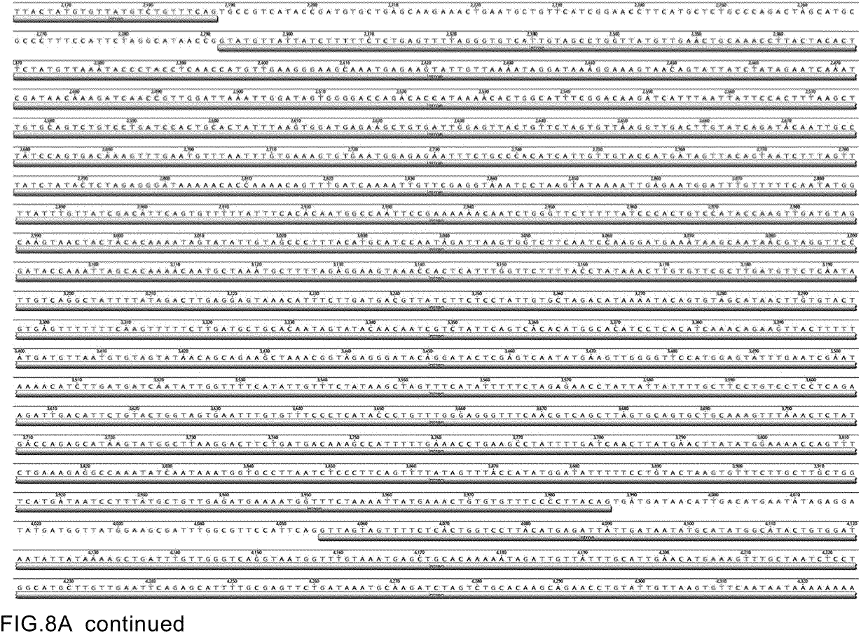

A CPP-fused Cas9 (Cas9-H8, Cas9-H12, Cas9-H16, or Cas9-H20) (20 µM) dissolved in SEC Buffer (20 mM HEPES-KOH, 500 mM KCl, pH 7.5) and a gRNA (20 µM) dissolved in Duplex Buffer (30 mM HEPES-KOH, 100 mM potassium acetate, pH 7.5) were mixed together in equal proportions. The mixture was incubated at room temperature for 15 minutes to prepare a gRNA + CPP-fused Cas9 complex (10 µM) . A gRNA + Cas9 complex (10 µM) was also prepared in a similar manner. Three gRNAs (gRNA1, gRNA2, and gRNA.3) were used (also in the following experiments). These gRNAs target particular sites (indicated as gRNA1 and gRNA2 in FIG. 2) in CjCHLI gene, which is Japanese cedar magnesium chelatase gene (having a nucleotide sequence set forth in SEQ ID NO: 1), and a particular site (indicated as gRNA3 in FIG. 8) in OsGW2 gene, which is rice E3 ubiquitin-protein ligase GW2 gene (having a nucleotide sequence set forth in SEQ ID NO: 2). The sites targeted by gRNA1 and gRNA2 represent bases at position 56 to 78 and position 1094 to 1116 in SEQ ID NO: 1, respectively. The site targeted by gRNA3 represents bases at position 1796 to 1818 in SEQ ID NO: 2. gRNA1 and gRNA2 were designed with a known software program (ApE), and gRNA3 was designed with a known software program (CRISPRdirect or CRISPR-P2.0) .

### 5) Preparation of gRNA + Cas9 + CPP Complex (Preparation of a Complex to be Used for Peptide-Based Method)

Cas9 (20 µM) dissolved in SEC Buffer (20 mM HEPES-KOH, 500 mM KCl, pH 7.5) and a gRNA (20 µM) dissolved in Duplex Buffer (30 mM HEPES-KOH, 100 mM potassium acetate, pH 7.5) were mixed together in equal proportions. The mixture was incubated at room temperature for 15 minutes to prepare a gRNA + Cas9 complex (10 µM) . The resulting gRNA + Cas9 complex was then mixed with a K10G-CPP (K10GH8, K10H12, K10H16, or K10H20 peptide) (20, 200, or 2000 µM) dissolved in Duplex Buffer (30 mM HEPES-KOH, 100 mM potassium acetate, pH 7.5) in equal proportions. The mixture was incubated at room temperature for 60 minutes to prepare a gRNA + Cas9 + CPP complex. The gRNAs used were gRNA1, gRNA2, and gRNA3 as described above.

### 6) Genome Editing Test

One week after passage, 360 µL of Japanese cedar cells (20 mg/mL in ½ MD liquid medium) or 360 µL of rice cells (20 mg/mL in MS liquid medium) were placed in a 5 mL Falcon polystyrene round-bottom tube. Subsequently, 360 µL of Japanese cedar cells or 360 µL of rice cells in the tube were mixed with 40 µl of a gRNA + CPP-fused Cas9 complex or a gRNA + Cas9 + CPP complex prepared above. The mixture was then cultured at 25° C. for 24 to 72 hours while shaking at 120 rpm under protection from light. It was confirmed that fluorescently modified gRNA + CPP-fused Cas9 complexes or gRNA + Cas9 + CPP complexes were taken into Japanese cedar cells and rice cells under these experimental conditions.

Japanese cedar cells or rice cells after culture were collected by centrifugation (at 500 g for 10 min at 4° C.) . The collected cells were washed with ½ MD liquid medium more than once. Genomic DNA was extracted from the Japanese cedar cells or rice cells using a genomic DNA extraction kit for plant cells (DNAs-ioil-P kit, Rizo Inc.) and was used to amplify gene regions targeted for genome editing by PCR. The PCR products were subjected to amplicon sequencing analysis to determine whether the genome editing occurred or not. The PCR products were also cloned and subjected to Sanger sequencing analysis to determine whether the genome editing occurred or not.

### 7) Results

Genome editing (gene deletion) was detected in Japenese cedar cells subjected to recombinant protein-based method (treated with gRNA + CPP-fused Cas9 complexes) or in Japanese cedar cells subjected to peptide-based method (treated with gRNA + Cas9 + CPP complexes), in which the genome editing (gene deletion) was not detected in cells treated with a gRNA alone or a gRNA + Cas9 complex. Genome editing (gene deletion) was detected in rice cells subjected to recombinant protein-based method (treated with gRNA + CPP-fused Cas9 complexes), in which the genome editing (gene deletion) was not detected in cells treated with gRNA + Cas9 complexes.

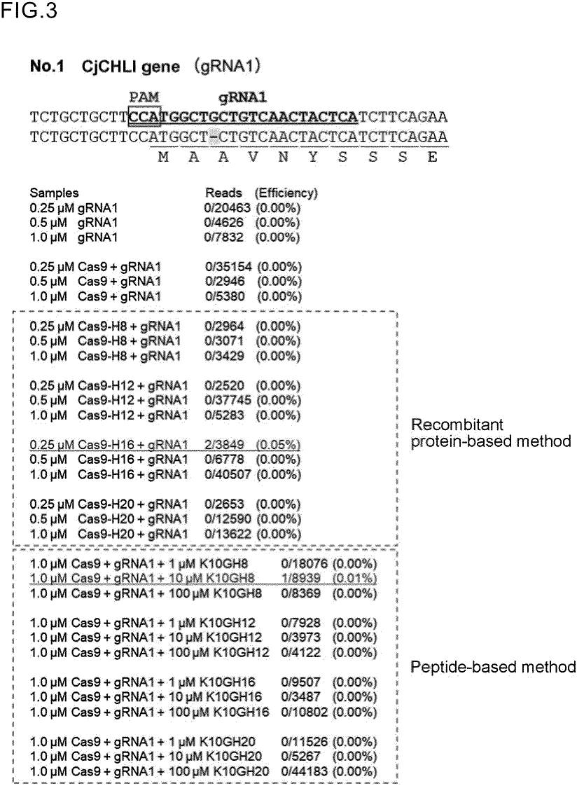

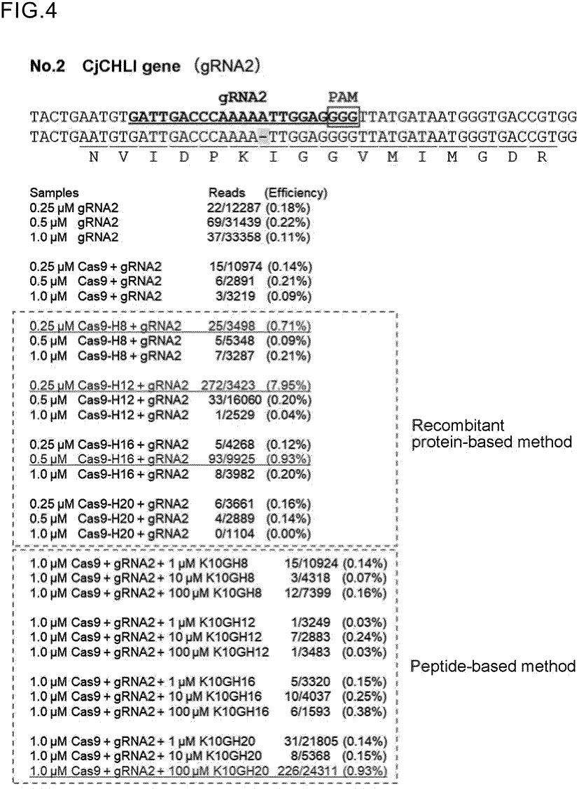

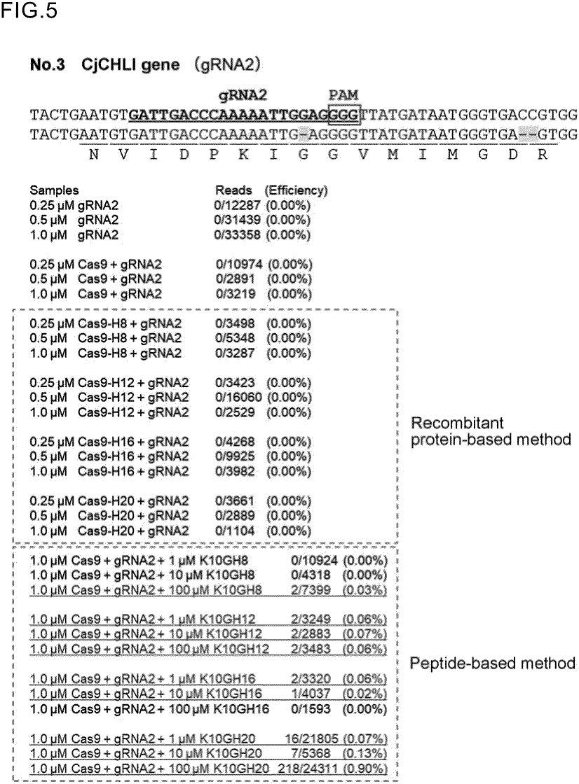

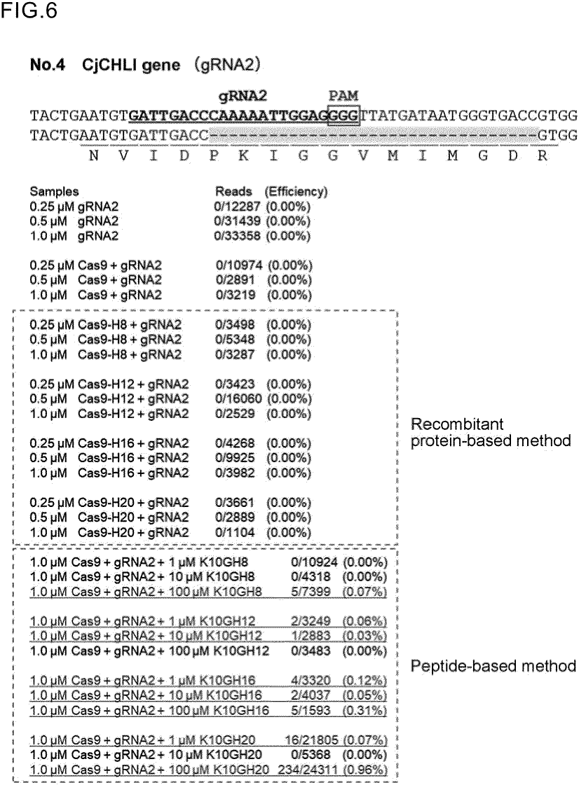

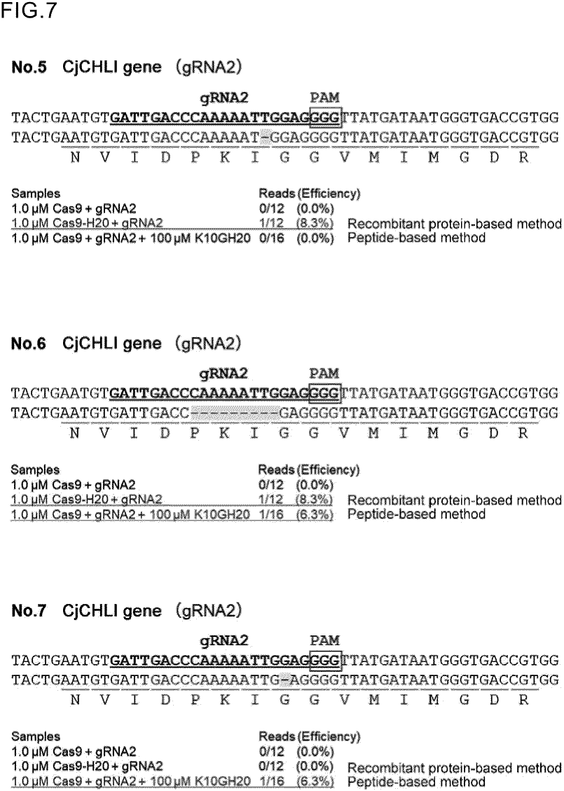

When either of two gRNAs (gRNA1 or gRNA2) corresponding to a target gene (CjCHLI gene) was used, four different genome-edited (gene-deleted) sequences were detected in recombinant protein-based method (gRNA + CPP-fused Cas9 complexes) (FIG. 3, FIG. 4, and FIG. 7) . In peptide-based method (gRNA + Cas9 + CPP complexes), six different genome-edited (gene-deleted) sequences were detected (FIG. 3, FIG. 4, FIG. 5, FIG. 6, and FIG. 7). In recombinant protein-based method (gRNA + CPP-fused Cas9 complexes), genome editing (gene deletion) was detected when four CPP-fused Cas9 proteins (Cas9-H8, Cas9-H12, Cas9-H16, and Cas9-H20) were used. In peptide-based method (gRNA + Cas9 + CPP complex), genome editing (gene deletion) was detected when four K10G-CPP peptides (K10GH8, K10GH12, K10GH16, and K10GH20 peptides) were used.

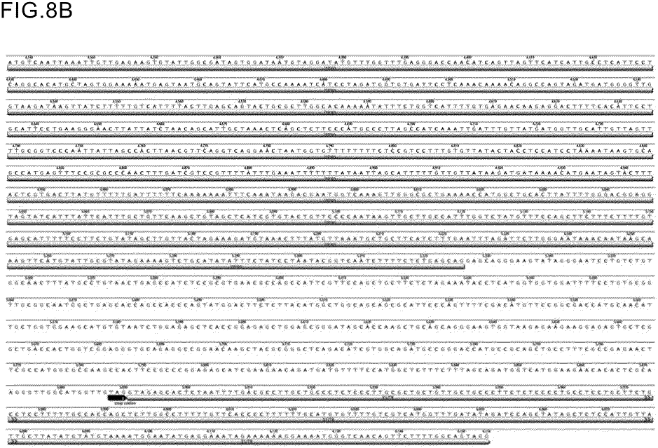

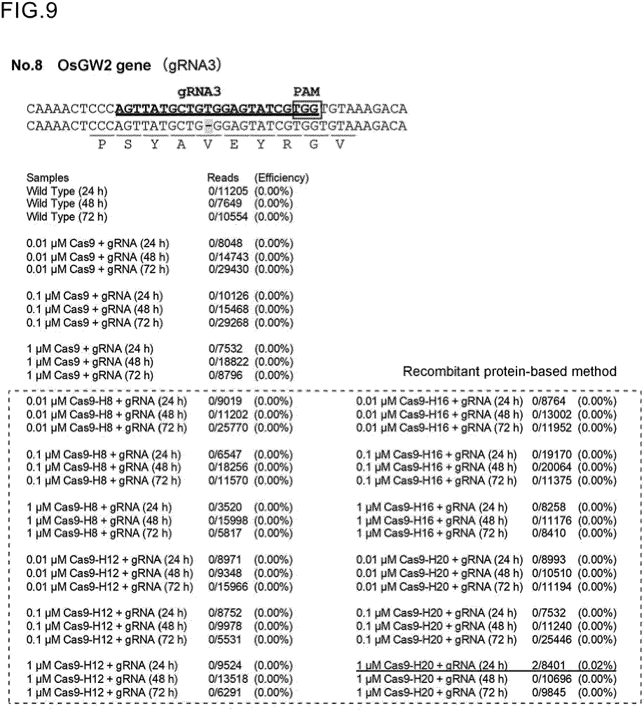

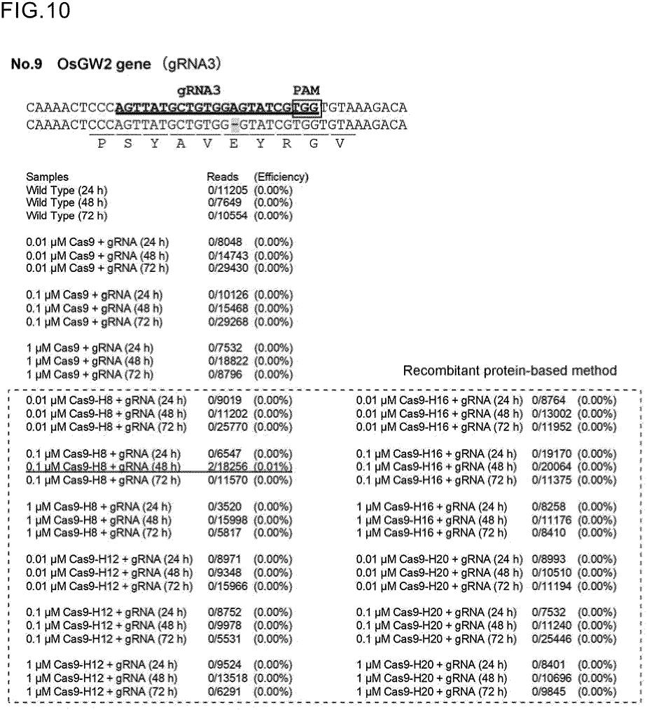

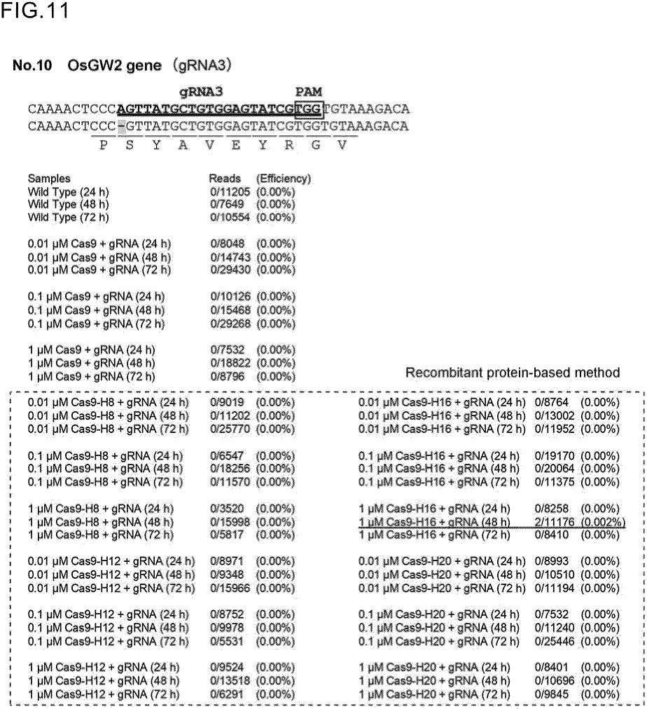

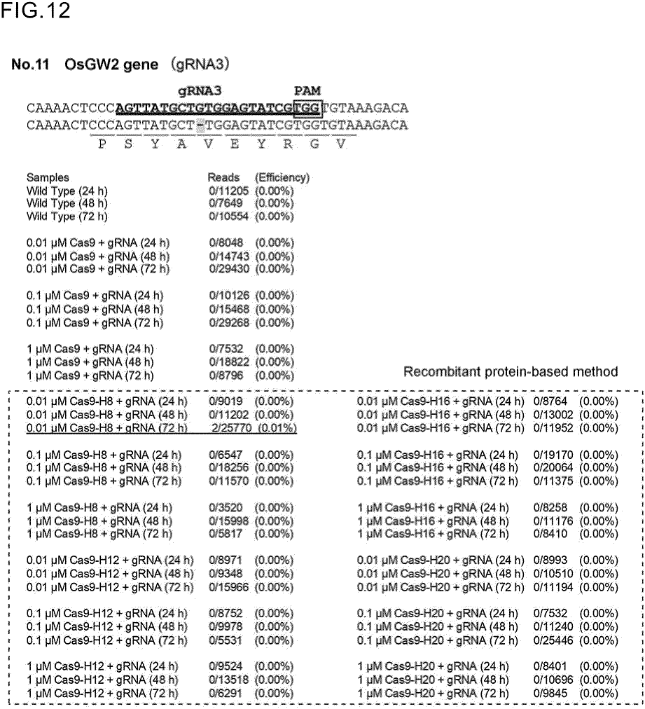

When gRNA3 corresponding to a target gene (OsGW2 gene) was used, five different genome-edited (gene-deleted) sequences were detected in recombinant protein-based method (gRNA + CPP-fused Cas9 complexes) (FIG. 8, FIG. 9, FIG. 10, FIG. 11, and FIG. 12). When three CPP-fused Cas9 proteins (Cas9-H8, Cas9-H16, and Cas9-H20) were used, genome editing (gene deletion) was detected in recombinant protein-based method (gRNA + CPP-fused Cas9 complexes).

Regions where the genome editing (gene deletion) was detected matched to regions recognized by the gRNAs, confirming that genome editing was induced in target regions.

These results demonstrate the effect of genome editing using the complexes of the present invention. In other words, these results demonstrate that utilizing the complexes of the present invention enables transfer of genome editing enzymes, which are difficult to directly transfer into plant cells, and induction of genome editing.

Utilizing the present invention achieves cultivar improvement of crops without relying on gene recombination. Crops obtained from the present invention are not considered as genetically modified organisms and thus are commercially valuable. In particular, cultivar improvement with a genome editing technique relying on conventional gene recombination has been impracticable in Japanese cedar, which was used in the Examples of the present invention, because the crop has an extremely long generation time and because removal of a foreign gene (a gene encoding a genome editing enzyme Cas9) by cross fertilization requires tens of years. The present invention is also expected to be extremely effective in genome editing (molecule breeding, such as cultivar improvement, by utilizing genome editing) of such crops having long generation time.

### INDUSTRIAL APPLICABILITY

The present invention is applicable to fields such as agriculture, forestry, food, pharmaceuticals, and study, breeding, and cultivar improvement of plants.

1. A complex comprising a genome editing enzyme and a cell membrane-permeable peptide (CPP), wherein the CPP is fused to the genome editing enzyme.

2. A complex comprising a genome editing enzyme, a target gene-specific nucleic acid, and a CPP, wherein the CPP is fused to the genome editing enzyme and/or the target gene-specific nucleic acid.

3. The complex according to claim 1, wherein the CPP is covalently attached to the genome editing enzyme and/or the target gene-specific nucleic acid.

4. The complex according to claim 3, wherein the CPP is covalently attached to the genome editing enzyme.

5. The complex according to claim 2, wherein a polycationic moiety is fused to the CPP and is electrostatically attached to the target gene-specific nucleic acid.

6. The complex according to claim 5, comprising a polycationic moiety that is covalently attached to the CPP.

7. The complex according to claim 6, wherein the polycationic moiety is a polycationic peptide.

8. The complex according to claim 7, wherein the polycationic peptide comprises ten or more lysine residues or ten or more arginine residues.

9. The complex according to claim 1, wherein 80% or more of the amino acid residues composing the CPP are histidine residues, and the CPP is eight to tens of amino acids in length.

10. The complex according to claim 9, wherein all of the amino acid residues composing the CPP are histidine residues.

11. The complex according to claim 1, further comprising a signal sequence.

12. The complex according to claim 1, further comprising a subdomain.

13. A method for genome editing, comprising transferring the complex according to claim 1 into a cell.

14. The method according to claim 13, wherein the cell is a plant cell, an algal cell, a filamentous fungal cell, or a yeast cell.

15. A kit for genome editing, comprising the complex according to claim 1 or components thereof.

16. The kit according to claim 15, wherein the kit is used for genome editing of a plant, an alga, a filamentous fungus, or a yeast.
